(19) United States
(12) Patent Application Publication
(10) Pub. No.: US 2005/0107876 A1
(43) Pub. Date: May 19, 2005

(54) Dermal substitute consisting of amnion and biodegradable polymer, the preparation method and the use thereof

(76) Inventors: Ki-Ho Kim, CheonAn (KR); Eun-Kyung Yang, CheonAn (KR); Sung-Po Kim, CheonAn (KR); Kang-Il Ko, CheonAn (KR); Jong-Won Lee, CheonAn (KR); Hyun-Mi Chang, CheonAn (KR); Jong-Ho Lee, Seoul (KR); Ki-Hwan Han, DaeGu (KR); Dae-Gu Son, DaeGu (KR)

Correspondence Address: David A. Einhorn, Esq.;Anderson Kill & Olick, P.C., 1251 Avenue of the Americas, New York, NY, 10020, US

(21) Appl. No.: 11/019,441

(22) Filed: Dec. 21, 2004

Related U.S. Application Data
(63) Continuation of application No. PCT/KR03/02012, filed on Sep. 30, 2003.

(30) Foreign Application Priority Data
Sep. 30, 2002 (KR) 2002-0059308
Sep. 18, 2003 (KR) 2003-0064761

(51) Int. Cl. A61F 2/10

(52) U.S. Cl. 623/15.12; 623/23.75; 435/396

(57) ABSTRACT

The present invention relates to a dermal substitute comprising the biodegradable polymer such as collagen and the biomaterial such as amnion, the preparation method and the use thereof. Specifically, the present invention provides with an amnion-collagen sponge complex structure prepared by attaching, inserting or incorporating an amnion obtained from placenta to/in collagen. Inventive dermal substitute can be applied to surgery and wound requiring skin graft, for example, severe burns such as second-degree burn, without rejection by immune system. Further, inventive dermal substitute with amnion instead of silicone membrane has several advantages, such as better biocompatibility, anti-inflammatory activity and promoting activity of wound healing and commercial utilization as basement membrane. Also, inventive complex structure can be used as the basic matrix of bio-artificial skin for culturing cells and the biodegradable basic matrix for preparing artificial organs.

### CROSS REFERENCE TO RELATED APPLICATION

This application is a continuation patent application of PCT Patent Application No. PCT/KR2003/002012, which was filed on Sep. 30, 2003, designating the United States of America, now abandoned.

### FIELD OF THE INVENTION

The present invention relates to the dermal substitute comprising the biodegradable polymer such as collagen and the like and the biomaterial such as amnion obtained from placenta, the preparation method and the use thereof.

### BACKGROUND OF THE INVENTION

Generally, in case that the skin damage below a second-degree burn is limited to the epidermis, the skin can be regenerated. But the injured dermis caused by severe burn is not recovered so that skin taken from one site of the patient is usually grafted to his burned area of the skin. Until the grafting, wounded area should be protected by covering material. Therefore, such these wound-covering materials should have the properties such as suitable water-permeability and protective activity from harmful environment such as microbes. For clinical applications, synthetic polymer membranes, for instance, urethane polymer, poly-L-leucine and the like, have been used as wound-covering materials with a limit to use, since they do not have any biological functions.

In order to regenerate the damaged skin more actively, several complex membranes comprising suitably permeable polymer membrane and biocompatible structure had been developed and designed to be detached as soon as the skin tissue was recovered. Biobrane, one of successful products in the early 1980's, has the bilayer structure consisting of collagen-covered nylon textile and micro-porous silicone membrane and shows many advantages, for example, the adhesiveness to skin, the expandability, the easy delivery of antibiotics to the burned area through silicone membrane to be commonly used for superficial burns, deep burns, after necrectomy on extensive granulating wound surface, on autografts, in donor regions, and after dermoabrasion And now, in the case of more severe wound over second-degree burn, dermal substitute is transplanted thereon and wounded area would be covered with partial thickness skin graft or cultured epidermal cell at 2 weeks after transplantation.

At that time, collagen in the form of sponge, gel or biodegradable polymers is used as a dermal substitute.

When the collagen sponge is applied to full thickness skin defected wound, fibroblasts and capillary blood vessels are infiltrated and proliferated in the sponge and collagen fibers are formed by self-collagen synthesis to build the dermis like-tissue. And then the collagen sponge is degraded and absorbed. Finally, the dermis like-tissue becomes complete self-dermal tissue. This collagen sponge is called as dermal substitute because the material itself turns to dermal tissue in the body. It is already commercially available and lots of studies and clinical reports have been published till now.

Korea patent publication No. 2000-0013701 discloses the porous, double structure wound protection membrane for inducing dermis-regeneration, which consists of porous layer and rigid layer comprising collagen, laminin and hyaluronic acid.

Korea patent publication No. 2000-0007983 discloses the biological tissue containing solidified collagen layer of complex structure comprising collagen sponge and mesh, and the use thereof.

Korean patent No. 94-1379 discloses tissue equivalent that is dermal substitute comprising collagen-grid form dermal layer wherein collagen layer was made by constructing fibroblastic cell Additionally Integra™ (USA) and Terudermis™ (Japan) allowed by FDA and KFDA as medical device are commercially available dermal substitute now. Those are consisting of silicone membrane synthetic polymer and collagen sponge layer. Upper layer is silicone membrane, preventing microbial infection and the evaporation of body fluid and the lower layer is collagen sponge layer, which induces the regeneration of the new blood vessel and the connective tissue, and is degraded and absorbed by itself in the body.

The treatment for severe burns using such commercialized dermal substitute usually requires 2 steps of grafting procedure; the first step is the formation of new dermis in 2-3 weeks by grafting dermal substitute on the wound; and the second step is the skin grafting for epidermis formation on the newly formed dermis. An above-mentioned conventional grafting procedure accompanies the long-term hospitalization and the pain of the patient. In order to solve the problems, dermal substitute grafting and autologous split-thickness skin graft/cultured epithelial autografts have been tried simultaneously, however, engraftment(take) rates of these grafts are not good since the present commericial product ; dermal substitute does not have the basement membrane.

Therefore, a certain material instead of basement membrane of skin has been need for increasing the rate of skin graftance till now.

Accordingly, there have been needed to develop biocompatible dermal substitute which allows one-step grafting procedure by autologous split-thickness skin graft(autograft) or cultured epithelial autografts simultaneous with dermal substitute grafting synchronized with promoting wound healing effect through increasing the rate of the skin graftance and anti-inflammation.

The present inventors had prepared a reinforced collagen sponge by inserting mesh type of collagen thread into the collagen sponge, disclosed in Korea patent publication No. 2000-7983. The disclosures of which are incorporated herein by references.

The present inventors have studied on the biocompatible amnion and have found the wound healing effect of amnion that prevents the infection, protects the loss of water, protein, etc. in the wound and facilitates epithelial cell migration to the wound by various kinds of growth and differentiation factors. And also the inventive dermal substitute prepared by attaching the amnion to collagen scaffold, makes it possible that one-step grafting procedure is enough to complete grafting, not conventional two steps required grafting.

Finally, the present inventors have endeavored to develop the bioartificial skin using amnion as a basement membrane and completed the present invention by deveolping inventive dermal substitute comprising amnion and biodegradable polymer.

### SUMMARY OF THE INVENTION

The present invention provides the dermal substitute comprising the biodegradable polymer such as collagen and the like and the biomaterial such as amnion obtained from placenta, the preparation method and the use thereof.

### BRIEF DESCRIPTION OF THE DRAWINGS

The above and other objects, features and other advantages of the present invention will more clearly understood from the following detailed description taken in conjunction with the accompanying drawings, in which;

FIG. 1 shows the scanning electron microscopy of porous collagen sponge; FIG. 1a is the sectional view thereof and FIG. 1b is the bottom view thereof;

FIG. 2 shows the scanning electron microscopy of inventive amnion-collagen sponge complex structure; FIG. 2a is the attached part between amnion and collagen sponge of inventive dermal substitute prepared in Example 1; and FIG. 2b is that of inventive dermal substitute prepared in Example 2;

FIG. 3a is the picture of H&E stained tissue section observed at 1 month after transplanting only collagen sponge in the intra-stroma of rabbit cornea; FIG. 3b is the picture of infiltrated inflammatory cells around the implant; and FIG. 3c is the picture of H&E stained tissue section observed at 1 month after transplanting inventive amnion-collagen sponge complex in the intra-stroma of rabbit cornea;

FIG. 4a is the picture of H&E stained tissue section observed at 1 week after transplanting only collage sponge in the back of nude mouse; and FIG. 4b is the picture of H&E stained tissue section observed at 1 week after transplanting inventive dermal substitute, prepared by immersing collagen sponge into amnion extract, in the back of nude mouse;

FIG. 5a is the picture of H&E stained tissue section observed on 10 days after transplanting Terudermis™ in the full-thickness defected skin wound on the back of guinea pig; and FIG. 5b is the picture of H&E stained tissue section observed on 10 days after transplanting inventive amnion-collagen complex in the full-thickness defected skin wound on the back of guinea pig;

FIG. 6 shows the pictures on the 3, 7 and 30 days after transplant to the limbus of rabbit cornea, induced alkali-burns injury by N-heptanol (FIG. 6a, 6b and 6c; collagen sponge, FIG. 6d, 6e, 6f; inventive anmion-collagen sponge complex);

FIG. 7 shows the H&E stained tissue sections at 7 days after transplanting in the full-thickness wound by surgical excision on the back of New Zealand white rabbit; FIG. 7a is for only collagen sponge, FIG. 7b is for collagen sponge and autologous split-thickness skin graft simultaneously and FIG. 7c is for inventive amnion-collagen sponge complex and autologous split-thickness skin graft simultaneously;

FIG. 8 is the standard curve of EGF presenting the relation of EGF amount and absorbance;

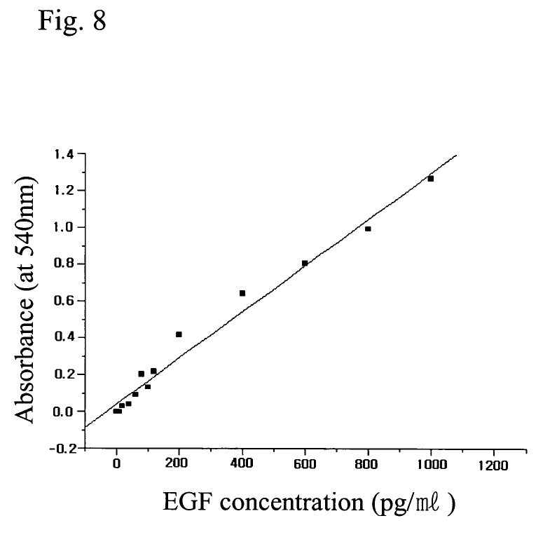

FIG. 9 is the immunohistochemical staining picture of basement membrane component of amnion; FIG. 9a is for EGF-receptor in amnion; and FIG. 9b is for collagen type IV in anmion;

FIG. 10 is the immunohistochemical analysis of bio-artificial skin for cytokeratin.

### DETAILED DESCRIPTION OF THE INVENTION

Accordingly, it is an object of the present invention to provide a dermal substitute comprising complex of the biodegradable polymer structure and the biocompatible amnion which have a major role of basement membrane, the preparation method and the use thereof.

The term “complex” herein comprises the attached form, incorporated form or inserted form of biodegradable polymer and amnion.

The term “biodegradable polymer” herein comprises at least one selected from the group consisting of natural materials such as collagen, gelatin, hyaluronic acid and its derivatives, chitin, chitosan, alginate, fibronectin and dextran; synthetic materials such as PLGA (poly(D,L-lactic-co-glycolic acid)), PGA (polyglycolic acid), PLA (poly(lactic acid)) and copolymer analog thereof, poly ε-caprolactone, polyanhydride, polyorthoesters, polyurethane and the like.

The structure of biodegradable polymer herein is preferably in the form of sponge, film, fiber and the like.

Above described amnion herein can be prepared by procedure comprising the steps: preparing sheet structure using double ring, insert or silicone ring, mesh structure or an amnion extract and subsequent attaching, incorporating or inserting said anmion onto/into biodegradable polymer.

The terms “attached” and “inserted” herein means that amnion and biodegradable polymer is in physically and closely contacted condition each other or any of them is entered into between layered structures to bind each other. The term “incorporated” herein means that one or both of amnion and biodegradable polymers are bound each other by mixing, for example, immersing in other structure.

The inventive dermal substitute can be prepared by various methods as follows; 1) by attaching the amnion to the biodegradable polymer sponge in the process of polymer structure manufacturing step; 2) by attaching the amnion to the biodegradable polymer sponge after polymer structure manufacturing process comprising cross-linking; or 3) by immersing the biodegradable polymer sponge into amnion extract.

Hereinafter, the present invention is described in detail.

The present invention provides improved treating and healing effect and the increased convenience to use the product by introducing amnion into inventive dermal substitute, which is attached, incorporated or inserted to/in/into biodegradable polymer substrate, a conventional and artificial substrate.

The biodegradable polymer of the present invention should be as a backbone to which cells can be attached easily for three-dimensional structure; be immunologically inactive in order not to provoke an inflammatory response or a foreign substance response; be able to react actively with surrounding tissues and induce the neighboring cells to grow/proliferate into the structure of itself; have proper degradation rate since it has to act as a supporting layer not to be degraded quickly by foreign body response after grafting; and be completely degraded and disappeared by itself in a certain period.

Accordingly, the polymer of the present invention comprises all the materials which can be degraded in the body. In the preferred embodiment of the present invention, collagen, a kind of structural protein in the body, can be used as a biodegradable polymer.

The inventive dermal substitute can be prepared by various methods as follows; 1) by attaching the amnion to the biodegradable polymer sponge in the process of polymer structure manufacturing step; 2) by attaching the amnion to the biodegradable polymer sponge after polymer structure manufacturing process comprising cross-linking; or 3) by immersing the biodegradable polymer sponge into amnion extract.

In order to manufacture the inventive dermal substitute having complex structure formed by attaching the amnion to the biodegradable polymer sponge in the process of polymer structure manufacturing step, the present invention provides the method of preparing dermal substitute having complex structure comprising biodegradable polymer and amnion, which is characterized in comprising steps consisting of (step 1) dissolving collagen fiber in acidic solution, preferably acetic acid or pepsin, at the concentration ranging from 0.3 to 1% and pH ranging from 3 to 4; (step 2) spreading the solution prepared in step (1) on the amnion-attached mold (i.e. 12-well plate); (step 3) freezing the mold in the refrigerator at the temperature ranging from −196° C. to 0° C. and then freeze-drying over 36 hours.

Also, the present invention provides the dermal substitute manufactured by above-described method, having complex structure attaching the amnion to the biodegradable polymer sponge in the process of polymer structure manufacturing step.

To increase the biodegradability and tensile strength, the present invention provides the method of preparing dermal substitute having amnion-collagen sponge complex structure prepared by comprising crosslinking collagen and amnion in conventional crosslinking manner well known in the art.

For example, conventional crosslinking method comprises 0.25% glutaraldehyde treatment, 33 mM 1,3-carbodiimide and 6mM hydroxysuccinimide (dissolved in 90% acetone) treatment, 33 mM 1,3-carbodiimide and 6 mM hydroxysuccinimide (dissolved in 40% alcohol) treatment, UV and gamma irradiation, other chemical crosslinking methods and so on.

In order to manufacture the inventive dermal substitute having complex structure formed by attaching the amnion to the biodegradable polymer sponge after its manufacture process comprising cross-linking, the present invention provides the method of preparing dermal substitute having complex structure comprising biodegradable polymer and amnion, which is characterized in comprising the steps consisting of (step 1) dissolving collagen fiber in acid solution, preferably acetic acid or pepsin, at the concentration ranging from 0.3 to 1% about pH ranging from 3 to 4; (step 2) spreading the solution prepared in (step 1) on the mold (i.e. 12-well plate); (step 3) freezing the mold in the refrigerator at the temperature ranging from −196° C. to 0° C. and then freeze-drying over 36 hours; (step 4) performing DHT(dehydrothermal) crosslinking that comprises the steps consisting of putting collagen sponge into the vaccum oven, maintaining in vacuo at the room temperature for 2 hours and at 110° C. for 24 hours and removing vacuum at 30° C.; (step 5) performing crosslinking collagen according to above conventional crosslinking method repeatedly; (step 6) coating the crosslinked-collagen sponge or amnion with 0.01% to 0.05% of collagen solution; (step 7) attaching collagen sponge to amnion; (step 8) freezing the complex structure in the refrigerator at the temperature ranging from −196° C. to 0° C. and then freeze-drying over 36 hours.

The pore size of which aforementioned collagen sponge ranges from 40 to 150 μm, preferably 60 to 120 μm.

Also, the present invention provides the dermal substitute manufactured by above-described method, having complex structure attaching the amnion to the biodegradable polymer sponge after its manufacture process.

In order to manufacture the inventive dermal substitute having complex structure formed by immersing the biodegradable polymer sponge into the amnion extract, the present invention provides the method of preparing dermal substitute having complex structure comprising biodegradable polymer and anmion, which is characterized in comprising the steps consisting of ;(step 1) dissolving collagen fiber in acid solution, preferably acetic acid or pepsin, at the concentration ranging from 0.3 to 1% about pH ranging from 3 to 4; (step 2) spreading the solution prepared in (step 1) on the mold (i.e. 12-well plate); (step 3) freezing the mold in the refrigerator at the temperature ranging from −196° C. to 0° C. and then freeze-drying over 36 hours; (step 4) performing DHT(dehydrothermal) crosslinking that comprises the steps of putting collagen sponge into the vaccum oven, maintaining in vacuo at the room temperature for 2 hours and at 110° C. for 24 hours and removing vacuum at 30° C.; (step 5) performing again crosslinking collagen according to above conventional crosslinking method; (step 6) preparing the amnion extract; (step 7) immersing the collagen sponge into amnion extract over 12 hours; (step 8) freezing the complex structure in the refrigerator at the temperature ranging from −196° C. to 0° C. and then freeze-drying over 36 hours.

Above-mentioned amnion extract of step 6 can be prepared by pulverizing and homogenizing a freeze-dried amnion, centrifuging and collecting the supernatant and then filtrating them.

As the inventive amnion-collagen sponge dermal substitute was grafted to intra-stroma of rabbit cornea, it showed more potent anti-inflammatory activity by inhibiting the infiltration of inflammatory cell than in case of the grafting with collagen sponge only. Also in the transplant of the inventive collagen sponge immersed into the amnion extract into the subcutaneous tissue of the back of a nude mouse, it causes less inflammatory response and delayed collagen degradation to provide endurance than control.

In grafting commercially available Terudermis and the inventive anmion-collagen sponge dermal substitute in the back of guinea pig having full-thickness skin defected wound, showed the new blood vessel formation and fibroblast proliferation in both of them and especially, the inventive dermal substitute shows excellent effect on re-epithelialization.

In grafting the inventive amnion-collagen sponge dermal substitute after n-heptanol treatment at the limbus of rabbit, it showed anti-inflammatory effect by inhibiting the infiltration of inflammatory cells, differently from the grafting collagen sponge.

The amnion in the present dermal substitute can be prepared by freezing or freeze-drying treatment, however the EGF (epidermal growth factor) therein are not so much decreased that the inventive dermal substitute can express its wound-healing facilitating activity.

The dermal substitute of the present invention using amnion instead of silicone membrane has the good biocompatibility, the high engraftment(take) rates of autologous split-thickness skin graft because of providing basement membrane, the anti-inflammatory activity, and wound-healing facilitating activity so it would be useful for as wound dressing for healing.

Also, the present invention provides the dermal substitute prepared by culturing cell in amnion-biodegradable polymer complex structure manufactured in accordance to the above-described method.

Above-described cell which can be cultured in the polymer structure comprises at least one selected from the group consisting of fibroblast, keratinocytes, chondrocyte, osteocyte, muscle cell, oral mucosal cell, cornea stem cell and so on.

The present invention provides the bio-artificial skin, and the preparation thereof, which is characterized in culturing epithelial or epidermal, cells repeatedly on the amnion of bio-artificial dermis obtained from culturing stromal or dermal cells in anmion-biodegradable polymer sponge complex.

Above-described cell which can be cultured in the polymer sponge comprises at least one selected from the group consisting of fibroblast, keratinocytes, chondrocyte, osteocyte, muscle cell, oral mucosal cell, cornea stem cell and so on.

By using an amnion as the basement membrane of bio-artificial skin, the problems occurring in conventional dermal substitute can be overcome.

Since anmion itself acts as a basement membrane, the inventive dermal substitute as an alternative method can provide one-step grafting procedure to substitute with conventional dermal substitute requiring more than one grafting operation, and thereby it can give the pain relief and cost reduction of hospitalization to the patients.

The inventive artificial dermal substitute culturing human fibroblast can be used as supporter of bio-artificial skin required in the autologous split-thickness skin grafting or in the grafting of cultured epithelial cell from autologous/allogenic organism. The dermal substitute of the present invention can be used as substrate for the preparation of various artificial organs such as artificial skin, artificial cornea, artificial cartilage, artificial bone and artificial muscle etc.

For example, after culturing fibroblast in the biodegradable polymer scaffold of the present invention, autologous cell transplantation, culturing stem cell, adult cell and the like or using as a substrate for culturing immortalized cell can be subjected thereon.

When the inventive amnion-collagen sponge complex and autologous epidermis were transplanted in the back of New Zealand white rabbit, it showed the excellent skin graftance, no inflammation and the better formation & proliferation of fibroblast and blood vessel, comparing with those in the case of transplanting collagen sponge only or both of collagen sponge ad autologous epidermis was transplanted.

### BEST MODE FOR CARRING OUT THE INVENTION

It will be apparent to those skilled in the art that various modifications and variations can be made in the compositions, use and preparations of the present invention without departing from the spirit or scope of the invention.

The present invention is more specifically explained by the following examples. However, it should be understood that the present invention is not limited to these examples in any manner.

### EXAMPLES

The following Reference Example, Examples and Experimental Examples are intended to further illustrate the present invention without limiting its scope.

### Reference Example 1

### The Amnion Preparation 1

The placenta was serologically screened for guaranteeing its safety according to generally applicable rules of tissue bank. And the amnion separated from normal placenta was stored in 400 ml of sterile saline solution (0.9% NaCl) at 4° C. The amnion was washed 4 times for 10 mins with gentle shaking and was transferred to the fresh sterile saline solution to store at 4° C. overnight. The sponge layer of amnion hydrated was removed. Remaining amnion was washed 4 times with sterile saline for 10 mins and was prepared in the form of sheet or mesh to attach to collagen sponge. Mesh form thereof was manufactured by mesher or by perforating artificially.

### Reference Example 2

### The Amnion Preparation 2

The amnion extract was prepared by following procedure. The washed amnion of Reference Example 1 was fast-frozen in liquid nitrogen and then was crushed with a mortar and pestle. The crushed substance was homogenized and centrifuged at 6000 rpm for 30 mins. The supernatant thereof was filtered with ultrafiltration membrane (Centrikon Co.) to obtain the amnion extract used in the following experiment.

### Example 1

### Amnion-Collagen Sponge Complex Preparation 1

0.5% collagen (Matrixen-ASP, Bioland Ltd.) was prepared by dissolving in acetic acid or pepsin and was adjusted to pH 3.0. Collagen solution was vortexed by a homogenizer (Bead Beater, BioSpec Co.) at 1500 rpm for 5 mins. The 1.5 ml of cream type collagen solution was spread on the amnion-attached mold, i.e., 12-well plate and the complex structure was frozen in the refrigerator at the temperature ranging from −196° C. to 0° C. and then freeze-dried over 36 hours.

To increase the biodegradability and tensile strength, the above-prepared amnion-collagen sponge complex structure was subjected to the conventional crosslinking procedure treated with 0.25% glutaraldehyde.

### Example 2

### Amnion-Collagen Sponge Complex Preparation 2

0.5% collagen (Matrixen-ASP, Bioland Ltd.) was prepared by dissolving in acetic acid or pepsin and adjusted to pH 3.0. Collagen solution was vortexed by a homogenizer at 1500 rpm for 5 mins. The 1.5 ml of cream type collagen solution was spread on the amnion-attached mold, i.e., 12-well plate and the complex structure was frozen in the refrigerator at the temperature ranging from −196° C. to 0° C. and then freeze-dried over 36 hours.

Freeze-dried porous collagen sponge was put into the vaccum oven maintaining in vacuo at the room temperature for 2 hours to remove a trace amount of water and subsequently vacuum was sustained at the temperature up to 110° C. for 24 hours. After 24 hours, it was cooled down at 30° C. and the vacuum was removed.

Through above several steps, DHT (dehydrothermal treatment) crosslinking step was completed and it provides stabilizing its complex structure and suppressing its structural change.

And then second crosslinking treatment was performed. The collagen was crosslinked by conventional crosslinking method using 0.25% glutaraldehyde or 1,3-carbodiimide and then surface of collagen sponge was coated with 0.05% collagen solution.

The amnion of Reference Example 1 was attached thereon, at least one side of the collagen sponge and the complex structure was frozen in the refrigerator at the temperature ranging from −196° C. to 0° C. and then freeze-dried over 36 hours.

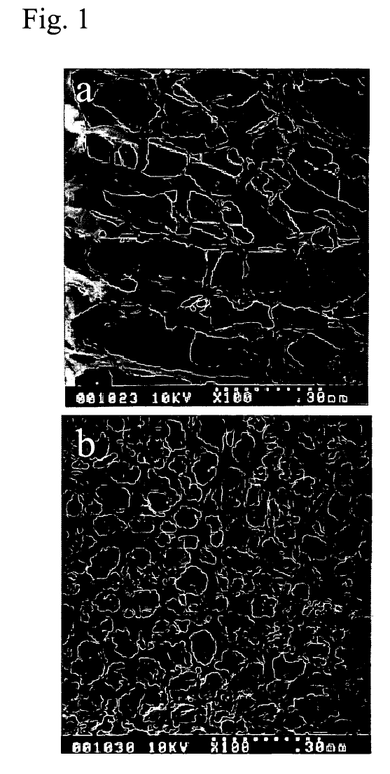

FIG. 1a and FIG. 1b represents the bottom and sectional view of conventionally manufactured-collagen sponge, respectively, observed by SEM (scanning electron microscope). The pore size thereof ranges from about 60 μm to 120 μm.

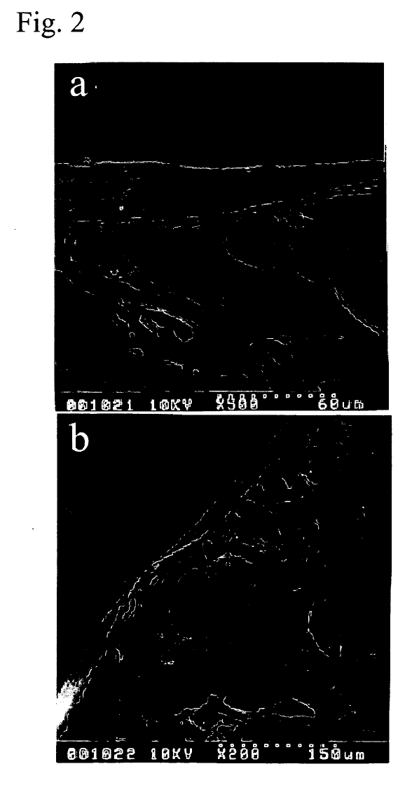

FIG. 2a and FIG. 2b shows the attached forms between amnion and collagen sponge in complex structures prepared in Example 1 and Example 2 of the present invention, respectively.

### Experimental Example 1

### Anti-Inflammatory Effect of Amnion-Collagen Sponge Complex Structure 1

To observe the degree of inflammation caused by implantation, the inventors carried out implantation of collagen sponge and inventive amnion-collagen sponge complex structure in the intrastroma of rabbit cornea and at 1 month after grafting, the tissue including graft was biopsied and subjected to the general procedures for histological observation; i.e.,fixed in formalin, washed, embedded in paraffin, sectioned with a 5 μm thickness, stained with H&E and examined under a light microscope.

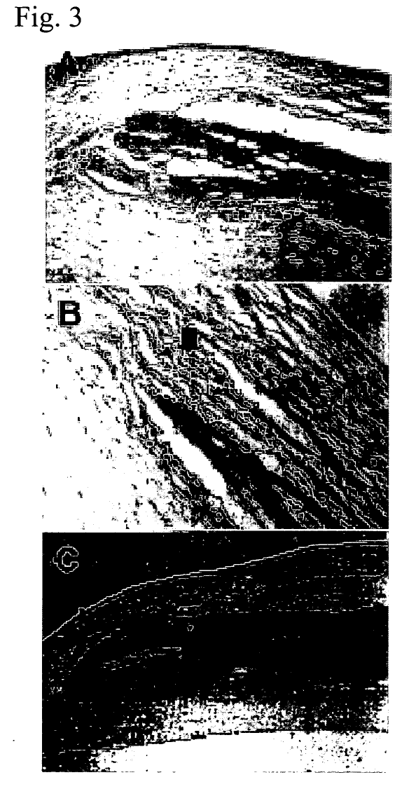

FIG. 3a depicts the picture of collagen sponge implanted in the intrastroma of rabbit cornea; FIG. 3b shows the ongoing infiltration of inflammatory cells around the implant at day 30 post-grafting and FIG. 3c shows that the inventive amnion-collagen sponge complex structure has the anti-inflammatory activity due to inhibiting activity from the infiltration of inflammatory cell.

### Experimental Example 2

### Anti-Inflammatory Effect of Amnion-Collagen Sponge Complex Structure 2

To confirm the anti-inflammatory activity of the amnion used in the present invention, the collagen sponge and the amnion extract-collagen sponge was implanted into the subcutaneous tissue of the back of a nude mouse and at 1 week after implantation, the biopsies were taken to examine the anti-inflammatory activity through H&E staining.

The collagen sponge was immersed in the amnion extract prepared in above Reference Example 2 for 24 hours and then used.

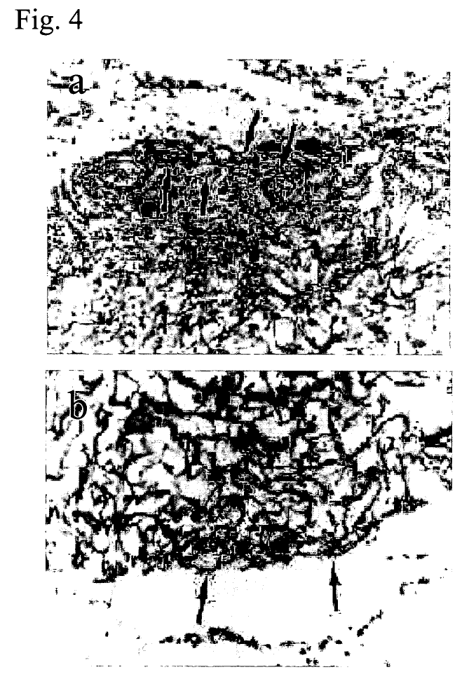

FIG. 4a presents the picture of collagen sponge-transplanted tissue; FIG. 4b depicts the picture of collagen sponge immersed in amnion extract, transplanted tissue, which leads less inflammatory response and maintains its original form without degradation after a week.

### Experimental Example 3

### Wound Healing Effect of Amnion-Collagen Sponge Complex Structure

To confirm the effect of inventive amnion-collagen sponge complex on wound healing, 350-400 g of guinea pig (Samtaco Co.) was injected intramuscularly with ketamine hydrochloride at a dose of 100 mg/kg bodyweight for anesthesia. The hair of the back was removed with electronic shaver. The back of pigs was washed and 10% povidone-iodine was applied thereon followed by swabbing with 70% ethanol.

Two 2 cm diameter-round full-thickness skin wounds were created on the back of each of guinea pig.

As a control group, Terudermis™ (Terumo Co., Japan) currently used for artificial dermis, was transplanted on the one side of wound and inventive anmion-collagen sponge complex of Example 1 was transplanted on the other side of wound. Subsequently, the wounds were covered with polyurethane film and fixed with elastic bandage. At 10 days after transplant, the biopsies were taken and sectioned to examine the wound healing through H&E staining.

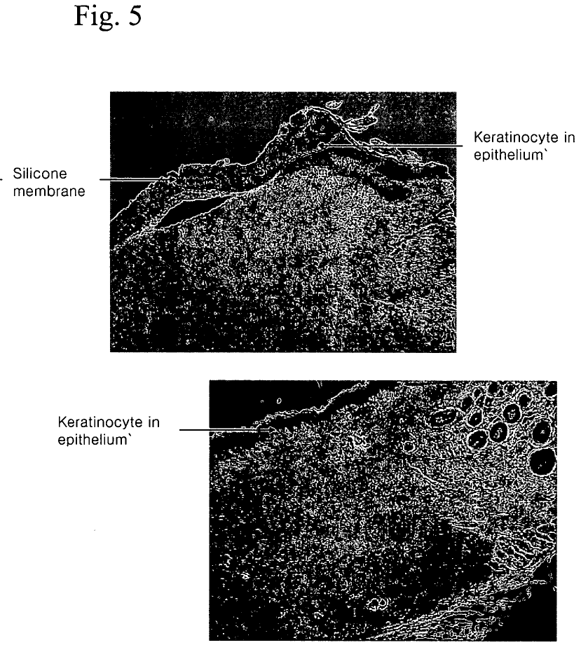

FIG. 5a and FIG. 5b showed the pictures of histological observation of Terudermis-transplanted one and inventive dermal substitute-transplanted one, respectively, 10 days after transplant. In both of them, the new blood vessel formation and fibroblast proliferation was observed and the collagen synthesis was augmented.

Additionally, the keratinocyte migration under silicone membrane was not observed in FIG. 5a for control, but the cell migration was detected in FIG. 5b for experimental group using inventive dermal substitute with amnion, which brought fast re-epithalization of epidermis.

### Experimental Example 4

### Wound Healing Effect of Amnion-Collagen Sponge Complex Structure

After n-heptanol treatment, alkali-burn at the limbus of rabbit in order to form new blood vessel in cornea, collagen sponge and inventive amnion-collagen sponge complex was transplanted. At each 3, 7 and 30 days, the tissue transplanted each dermal substitute was examined to compare its wound healing effect according to the time.

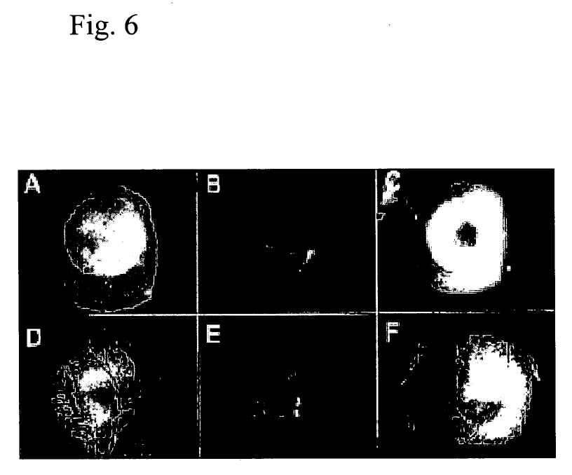

FIG. 6a, 6b and 6c are the pictures of collagen sponge-transplanted tissue at day 3, day 7 and day 30, respectively.

FIG. 6d, 6e and 6f are the pictures of inventive amnion-collagen sponge complex-transplanted tissue at day 3, day 7 and day 30, respectively.

Comparing with collagen sponge-transplanted tissue, amnion-collagen sponge complex suppressed over-growth of blood vessel as corneal injury at 30^{th} day after transplant and was less degraded.

### Experimental Example 5

### Effect of Amnion-Collagen Sponge Complex Structure on Engraftment

For autologous split-thickness skin graft(autograft), the engraftment (take) rates and wound healing effect of amnion were examined by transplanting the inventive amnion-collagen sponge complex and autograft simultaneously.

Three 4 cm-diameter round full-thickness skin wounds were created on the back of 2.4 kg of New Zealand white rabbit (Samtaco Co.) by surgical excision.

Collagen sponge was transplanted on the one side of wound, followed by transplanting 0.25 mm thickness of autograft using dermatome and inventive amnion-collagen sponge complex of Example 2 was transplanted on another side of wound, followed by same autografting procedure as described above.

As a control group, only collagen sponge was transplanted without autografting.

At 7 and 14 days after transplant, the biopsies were taken and stained with hematoxyline & eosin to observe the degree of wound healing.

The criteria evaluating wound healing includes skin graftance, inflammatory cell number, new vascularization and fibroblast proliferation under light microscope.

The evaluation results were classified into 4 groups, i.e., +, ++, +++ and ++++.

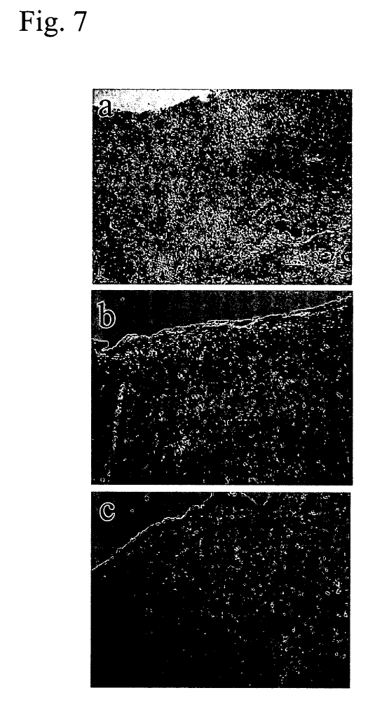

FIG. 7a shows the control transplanted with only collagen sponge; FIG. 7b shows the group transplanted with collagen sponge and autograft simultaneously; FIG. 7c shows the group transplanted with inventive amnion-collagen sponge complex and autograft simultaneously.

At the results of histological examination, the inventive amnion-collagen sponge complex showed the excellent engraftment of transplated epidermis, however, epidermis in the group transplanted with collagen sponge and autograft was partially sloughed off.

Accordingly, it was confirmed that the amnion in transplanted region was biodegraded without any inflammation in 2 weeks.

Moreover, as can been seen in Table 1, the fibroblast and blood vessel endothelial cell was actively infiltrated into complex structure in case that the inventive dermal substitute and autograft were simultaneously transplanted.

TABLE 1

| | | Control | Collagen | |
|---|---|---|---|---|
| | | (Collagen sponge | sponge/ | Amnion-collagen |
| | Day | only) | autograft | sponge/autograft |
| Skin graftance | 7 | − | + | ++++ |
| (splitthickness) | 14 | − | − | +++ |
| Inflammation | 7 | ++ | ++ | ++ |
| | 14 | + | + | + |
| New | 7 | + | + | +++ |
| vascularization | 14 | ++ | ++ | ++++ |
| Fibroblast | 7 | + | ++ | +++ |
| proliferation | 14 | ++ | +++ | ++++ |

*Inflammation (the number of infiltrated leukocytes): 0˜10(+), 10˜100(++), 100-500(+++), >500(++++)
*New vascularization (the number of lumen lined with endothelial cells): 0˜10(+), 10˜20(++), 20˜30(+++), >30(++++)
*Fibroblast proliferation (the number of proliferated fibroblasts): 0˜100(+), 100˜500(++), 500-1000(+++), >1000(++++)

### Experimental Example 6

### Measurement of Epidermal Growth Factor in the Amnion

The amnion was prepared from the placenta obtained by Cesarean section in 12 hours. The amnion was incubated in TSA medium (Tryptic soybean casein digest medium) for at least 7 days at 37° C. in order to determine whether microorganism remains or not.

After the incubation, the microorganism-detected amnion was discarded.

The guaranteed amnion was washed 4 times with about 400 ml sterile saline(0.9% NaCi) for 10 mins with shaking. The washed amnion was stored in fresh sterile saline overnight at 4° C. Thereby, the hydrated amnion sponge layer was eliminated and the amnion was washed again 4 times with about 400 ml sterile saline(0.9% NaCi) for 10 mins with shaking.

The amnion prepared by the procedure disclosed in above was frozen and freeze-dried prior to the extraction with PBS to obtain the amnion extract. The amnion extract was centrifuged for 5 mins at 15,000 rpm and the supernatant thereof was used to measure the amount of epidermal growth factor (EGF) using EGF detection kit (Asan pharmaceuticals Co.). According to EGF (Koma Biotech Co.) standard curve at 540 nm, EGF amount in each sample was determined (See Table 2).

TABLE 2

| | Untreated | Deep frozen | Freeze-dried |
|---|---|---|---|
| | (pg/mg) | (pg/mg) | (pg/mg) |
| Sample 1 | 3.02 | 2.2 | 2.31 |
| Sample 2 | 2.53 | 2.27 | 2.54 |
| Sample 3 | 3.89 | 3.4 | 3.7 |

As can bee seen in Table 2, the amnion processes including deep freezing, freeze-drying etc. did not reduce the EGF amount in amnion and rather maintained unchangeably.

Therefore, it is possible to anticipate that the EGF in amnion facilitates the wound healing.

### Experimental Example 7

### Immunohistochemical Staining on the Amnion

To determine the location of EGF receptor (EGF-R) and collagen type IV, a major component of basal lamina, immunohistochemical staining was performed with EGF-R antibody (DAKO Co., K0675) and collagen type IV antibody (NeoMarkers Co., MS-747-S1) according to immuno-peroxidase method well known in the art.

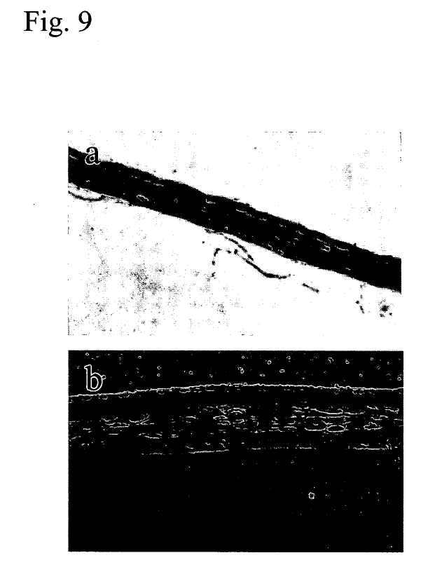

In FIG. 9a, the dark brown area is for the EGF-R; in FIG. 9b, the dark brown area is for collagen type IV.

According to the results, the EGF-R and collagen type IV were strongly stained around basement membrane, in which epithelium of amnion exists.

### Example 3

### Full Thickness Bio-Artificial Skin Preparation using Amnion-Collagen Sponge Complex Structure

3-1. Artificial Dermis Preparation

3 cm-diameter round disk of dermal substitute, amnion-collagen sponge prepared in Example 2, was laid on the 3.5 cm-diameter cell culture dish. ˜3×10⁶ dermal cells/well were seeded thereon with 10% FBS/DMEM media and incubated in a 5% CO₂ incubator at 37° C. 5 hours after seeding, 2 ml of medium was added thereto and the medium was replaced with fresh one. After then, the cells were incubated for 7 days with changing medium every 2 days.

3-2. Full Thickness Bio-Artificial Skin Preparation

Keratinocytes were multi-layer cultured on basement membrane of amnion of artificial dermis prepared in Example 3-1 to manufacture an artificial skin.

Culture plate insert with 3 μm pore of polycarbonate membrane (millicell, Millipore Co.) was put in each well of 6-well plate. Cultivated and contracted artificial dermis was laid on the insert and ˜5×10⁵ of keratinocytes were seeded thereon in a 30 mm-diameter insert. 2 ml of serum free medium for keratinocytes was added to the inner side of the insert and 3 ml of medium was added to the outside thereof. If the tissue prepared as above is cultured in the medium for a week, keratinocytes grow to the extent of covering its surface. And after, the medium in the insert was removed and the medium was filled in the outside of the insert.

All the nutrient components and growth factors are delivered by the diffusion through dermis and it seems to be a similar circumstance to the epidermis of body. Consequently, keratinocytes were grown and differentiated on the border of the air and liquid medium for 2 weeks.

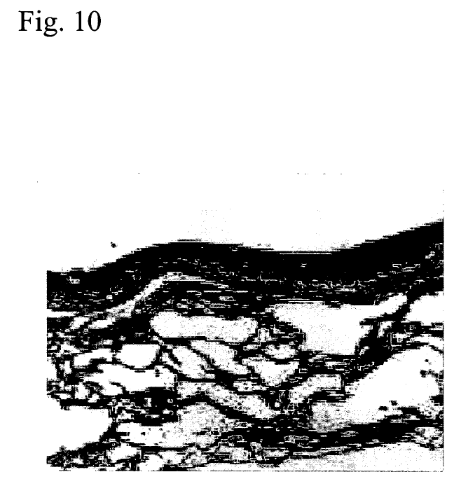

FIG. 10 shows the picture of immunohistochemical analysis of inventive full thickness bio-artificial skin.

The cytokeratin expressed in keratinocyte differentiation was determined according to the immuno-peroxidase method using cytokeratin antibody (Biomedical Technologies Co.) and it was confirmed that cytokeratin was detected through overall layer of keratinocyte.

1. A dermal substitute comprising the complex of biodegradable polymer structure and biocompatible amnion obtainable from placenta.

2. The dermal substitute according to claim 1 wherein said biodegradable polymer is at least one selected from the group consisting of natural materials such as collagen, gelatin, hyaluronic acid and its derivatives, chitin, chitosan, alginate, fibronectin and dextran; synthetic materials such as PLGA (poly(D,L-lactic-co-glycolic acid)), PGA (polyglycolic acid), PLA (poly(lactic acid)) and copolymer analog thereof, poly ε-caprolactone, polyanhydride, polyorthoesters, polyurethane and the like.

3. The dermal substitute according to claim 2 wherein said biodegradable polymer is collagen.

4. A method of preparing dermal substitute as set forth in claim 1 comprising complex of the biodegradable polymer structure and the biocompatible amnion obtained from placenta characterized in attaching, incorporating or inserting said amnion onto/into biodegradable polymer.

5. The method according to claim 4 wherein said biodegradable polymer is at least one selected from the group consisting of natural materials such as collagen, gelatin, hyaluronic acid and its derivatives, chitin, chitosan, alginate, fibronectin and dextran; synthetic materials such as PLGA (poly(D,L-lactic-co-glycolic acid)), PGA (polyglycolic acid), PLA (poly(lactic acid)) and copolymer analog thereof, poly ε-caprolactone, polyanhydride, polyorthoesters, polyurethane and the like.

6. The method according to claim 5 wherein said biodegradable polymer is collagen.

7. The method according to claim 4 wherein said biodegradable polymer is prepared in the form of sponge, film or fiber.

8. The method according to claim 4 wherein said amnion is prepared in the form of sheet structure, mesh structure or extract.

9. The method according to claim 8 wherein said sheet structure of amnion is made by using double ring, insert or silicone ring.

10. The method according to claim 4 wherein said amnion is attached to the biodegradable polymer sponge in the process of polymer structure manufacturing step.

11. The method according to claim 4 wherein said amnion is attached to the biodegradable polymer sponge after the process of polymer structure manufacturing step.

12. The method according to claim 4 wherein said biodegradable polymer sponge is immersed into amnion extract.

13. A method of preparing dermal substitute characterized in comprising the steps consisting of; preparing the amnion-biodegradable polymer sponge complex structure according to the method of claim 4; and culturing the cells thereon.

14. A dermal substitute prepared by the method as set forth in claim 13.

15. A method of preparing bio-artificial skin characterized in comprising the steps of; preparing the amnion-biodegradable polymer sponge complex structure according to the method of claim 4; culturing cells thereon; and culturing the cells on the amnion of said complex repeatedly.

16. The method according to claim 13 or claim 15 wherein said cell is at least one selected from the group consisting of fibroblast, keratinocytes, chondrocyte, osteocyte, muscle cell, oral mucosal cell and cornea stem cell.

17. A bio-artificial skin prepared by the method as set forth in claim 15.
